(19) United States
(12) Patent Application Publication
(10) Pub. No.: US 2010/0210596 A1
(43) Pub. Date: Aug. 19, 2010

(54) USE OF AMINOPYRIMIDINE COMPOUNDS IN THE TREATMENT OF IMMUNE DISORDERS

(76) Inventors: Jeffrey A. Bluestone, San Francisco, CA (US); Arthur Weiss, Mill Valley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/161,914

(22) PCT Filed: Jan. 29, 2007
(86) PCT No.: PCT/US07/02423

§ 371 Date: Jan. 13, 2009

Related U.S. Application Data
(60) Provisional application No. 60/764,492, filed on Feb. 1, 2006.

(51) Int. Cl. A61K 31/505; A61K 31/506; A61K 31/675; A61P 37/06

(52) U.S. Cl. 514/81; 514/256; 514/252.18; 514/275

(57) ABSTRACT

The present invention provides methods of treating immune disorders. The present invention provides methods of treating an autoimmune disorder; methods of reducing the risk of transplant rejection; methods of increasing transplant survival; and methods of treating graft-versus host disease. The methods generally involve administering to an individual in need thereof an effective amount of an aminopyrimidine compound.

### CROSS-REFERENCE

This application claims the benefit of U.S. Provisional Patent Application No. 60/764,492, filed Feb. 1, 2006, which application is incorporated herein by reference in its entirety.

### STATEMENT REGARDING FEDERALLY SPONSORED RESEARCH

The U.S. government may have certain rights in this invention, pursuant to grant no. R37 AI46643 awarded by the National Institutes of Health.

### BACKGROUND

The immune system protects the body from infectious agents and disease and is critical to our survival. However, in certain instances, the immune system can be the cause of illness. One example is in autoimmune disease wherein the immune system attacks its own host tissues, in many instances causing debilitating illness and sometimes resulting in death. A second example in which the immune system can cause illness is during tissue or organ transplantation. Except in the cases of genetically identical animals, such as monozygotic twins, tissue and organ transplants are rejected by the recipient's immune system as foreign. The immune reaction against transplants is even more pronounced in transplantation across species, i.e., xenotransplantation. Yet another example is graft-versus-host disease, in which transplanted cells mount an immune response against the transplant recipient's tissue.

An example of an autoimmune disease is Type 1 diabetes (T1D), or autoimmune diabetes. Autoimmune diabetes is mediated by the progression of a destructive T cell infiltration of insulin-producing islet β cells in the pancreas. Both CD4⁺ and CD8⁺ T cells cooperate in initiating insulitis as well as in islet β cell destruction via cytokines (IFNγ, TNFα) and direct cytolytic activity. Development of this pathogenic immunity is regulated by autoantigen presentation that requires IL-12-producing dendritic cells and B cells. Studies have shown that during the first 2 years of T1D there is a significant retention of insulin production. Maintaining this insulin secretion is an important clinical goal likely to reduce the risk of long term complications.

In order to inhibit detrimental immune responses that lead to transplant rejection and autoimmune disease, immunosuppressive drugs (such as cyclosporin A, tacrolimus, and corticosteroids) or antibody therapies (such as anti-T cell antibodies) are generally administered.

There is ongoing interest in developing methods for treating disorders resulting from detrimental immune responses.

### Literature

O'Hare et al. (2004) Blood 104:2532; O'Hare et al. (2005) Cancer Res. 65:4500; Burgess et al. (2005) Proc. Natl. Acad. Sci. USA 102:3395-3400; Martinelli et al. (2005) Haematologica 90:534-541; Veneri et al. (2005) New Engl. J. Med. 352:1049; Breccia et al. (2004) J. Clin. Oncol. 22:4653; Dietz et al. (2004) Blood 104:1094; Dewar et al. (2005) Immunology and Cell Biology 83:48-56; Zipfel et al. (2004) Curr. Biol. 14:1222; Gao et al. (2005) Leukemia 19:1905; Cwynarski et al. (2004) Leukemia 18:1332; Miyachi et al. (2003) Clin. Rheumatol. 22:329-332; Dewar et al. (2005) Cell. Cycle 4:851-853; Juurikivi et al. (2005) Ann. Rhem. Dis. 64:1126-1131; Bockelmann et al. (2005) Skin Pharmacol. Physiol. 18:42-54; Appel et al. (2005) Stem Cells 23:1082-1088; U.S. Pat. No. 6,986,116; Utset et al. (2002) J. Rheumatol. 29:1907-1913; Herold et al. (2005) Diabetes 54:1763-1769.

### SUMMARY OF THE INVENTION

The present invention provides methods of treating immune disorders. The present invention provides methods of treating an autoimmune disorder; methods of reducing the risk of transplant rejection; methods of increasing transplant survival; and methods of treating graft-versus host disease. The methods generally involving administering to an individual in need thereof an effective amount of an aminopyrimidine compound.

### BRIEF DESCRIPTION OF THE DRAWINGS

FIGS. 1A and 1B depict the effect of imatinib on blood glucose levels in diabetes-prone mice even after onset of disease. Importantly, the effects were maintained even after short term therapy.

FIG. 2 depicts diabetes incidence in NOD mice treated with Gleevec compared with control treated and age-matched NOD mice.

FIG. 3 depicts diabetes incidence in NOD mice injected with cyclophosphamide (CY) in conjunction with daily oral Gleevec therapy or control peanut oil.

FIG. 4 depicts the percent diabetic following Gleevec treatment in diabetic NOD mice treated with Gleevec for a short course (3 weeks) or longer courses (8-10 weeks) either daily or 3 times per week.

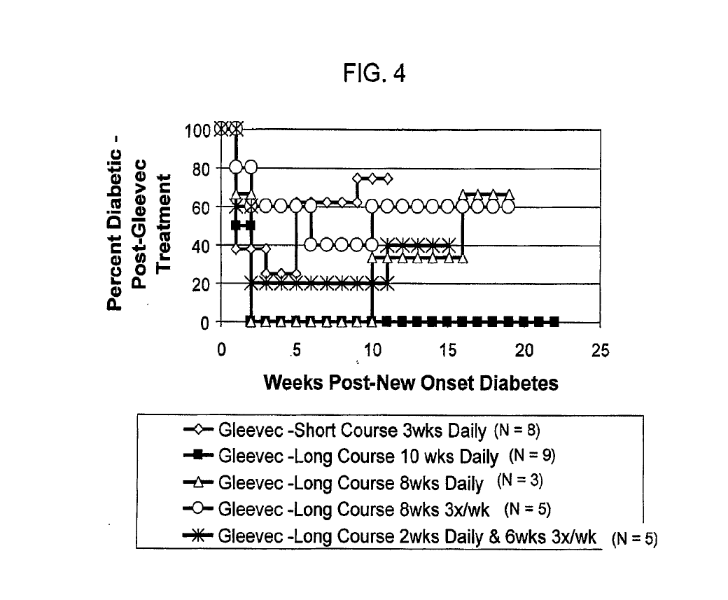

FIG. 5 depicts the results of a further exemplary dosing regimen of Gleevec on blood glucose levels.

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, particularly humans. Non-human animal models, particularly mammals, e.g. non-human primates, murines, lagomorpha, etc. may be used for experimental investigations.

The term “isolated compound” means a compound which has been substantially separated from, or enriched relative to, other compounds with which it occurs in nature. Isolated compounds are usually at least about 80%, at least 90% pure, at least 98% pure, or at least about 99% pure, by weight. The present invention is meant to comprehend diastereomers as well as their racemic and resolved, enantiomerically pure forms and pharmaceutically acceptable salts thereof.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

As used herein, “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and are either pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

A “pharmaceutically acceptable ester” of a compound means an ester that is pharmaceutically acceptable and that possesses the desired pharmacological activity of the parent compound, and includes, but is not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

A “pharmaceutically acceptable enol ether” of a compound means an enol ether that is pharmaceutically acceptable and that possesses the desired pharmacological activity of the parent compound, and includes, but is not limited to, derivatives of formula C═C(OR) where R is hydrogen, alkyl, alkenyl, alkynyl, aryl, heteroaryl, aralkyl, heteroaralkyl, cycloalkyl or heterocyclyl.

A “pharmaceutically acceptable enol ester” of a compound means an enol ester that is pharmaceutically acceptable and that possesses the desired pharmacological activity of the parent compound, and includes, but is not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl.

A “pharmaceutically acceptable solvate or hydrate” of a compound means a solvate or hydrate complex that is pharmaceutically acceptable and that possesses the desired pharmacological activity of the parent compound, and includes, but is not limited to, complexes of a compound with one or more solvent or water molecules, or 1 to about 100, or 1 to about 10, or one to about 2, 3 or 4, solvent or water molecules.

“Pro-drugs” means any compound that releases an active parent drug according to any one of the formulae described below in vivo when such prodrug is administered to a mammalian subject. Prodrugs of a compound of any one of the formulae described below are prepared by modifying functional groups present in the compound of a formula in such a way that the modifications may be cleaved in vivo to release the parent compound. Prodrugs include compounds of any one of the formulae described below wherein a hydroxyl, amino, or sulfhydryl group in the formula is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxyl functional groups in compounds of any one of the formulae described below, and the like.

The term “organic group” and “organic radical” as used herein means any carbon-containing group, including hydrocarbon groups that are classified as an aliphatic group, cyclic group, aromatic group, functionalized derivatives thereof and/or various combination thereof. The term “aliphatic group” means a saturated or unsaturated linear or branched hydrocarbon group and encompasses alkyl, alkenyl, and alkynyl groups, for example. The term “alkyl group” means a substituted or unsubstituted, saturated linear or branched hydrocarbon group or chain (e.g., C₁ to C₈) including, for example, methyl, ethyl, isopropyl, tert-butyl, heptyl, iso-propyl, n-octyl, dodecyl, octadecyl, amyl, 2-ethylhexyl, and the like. Suitable substituents include carboxy, protected carboxy, amino, protected amino, halo, hydroxy, protected hydroxy, nitro, cyano, monosubstituted amino, protected monosubstituted amino, disubstituted amino, C₁ to C₇ alkoxy, C₁ to C₇ acyl, C₁ to C₇ acyloxy, and the like. The term “substituted alkyl” means the above defined alkyl group substituted from one to three times by a hydroxy, protected hydroxy, amino, protected amino, cyano, halo, trifloromethyl, mono-substituted amino, di-substituted amino, lower alkoxy, lower alkylthio, carboxy, protected carboxy, or a carboxy, amino, and/or hydroxy salt. As used in conjunction with the substituents for the heteroaryl rings, the terms “substituted (cycloalkyl)alkyl” and “substituted cycloalkyl” are as defined below substituted with the same groups as listed for a “substituted alkyl” group. The term “alkenyl group” means an unsaturated, linear or branched hydrocarbon group with one or more carbon-carbon double bonds, such as a vinyl group. The term “alkynyl group” means an unsaturated, linear or branched hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polycyclic aromatic hydrocarbon group, and may include one or more heteroatoms, and which are further defined below. The term “heterocyclic group” means a closed ring hydrocarbon in which one or more of the atoms in the ring are an element other than carbon (e.g., nitrogen, oxygen, sulfur, etc.), and are further defined below.

“Organic groups” may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ethers, esters, haloalkyls, nitroalkyls, carboxyalkyls, hydroxyalkyls, sulfoalkyls, etc.

The terms “halo” and “halogen” refer to the fluoro, chloro, bromo or iodo groups. There can be one or more halogen, which are the same or different. Halogens of particular interest include chloro and bromo groups.

The term “haloalkyl” refers to an alkyl group as defined above that is substituted by one or more halogen atoms. The halogen atoms may be the same or different. The term “dihaloalkyl” refers to an alkyl group as described above that is substituted by two halo groups, which may be the same or different. The term “trihaloalkyl” refers to an alkyl group as describe above that is substituted by three halo groups, which may be the same or different. The term “perhaloalkyl” refers to a haloalkyl group as defined above wherein each hydrogen atom in the alkyl group has been replaced by a halogen atom. The term “perfluoroalkyl” refers to a haloalkyl group as defined above wherein each hydrogen atom in the alkyl group has been replaced by a fluoro group.

The term “cycloalkyl” means a mono-, bi-, or tricyclic saturated ring that is fully saturated or partially unsaturated. Examples of such a group included cyclopropyl, cyclobutyl, cyclopentyl, cyclohexyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like.

The term “(cycloalkyl)alkyl” means the above-defined alkyl group substituted for one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The term “substituted phenyl” specifies a phenyl group substituted with one or more moieties, and in some instances one, two, or three moieties, chosen from the groups consisting of halogen, hydroxy, protected hydroxy, cyano, nitro, trifluoromethyl, C₁ to C₇ alkyl, C₁ to C₇ alkoxy, C₁ to C₇ acyl, C₁ to C₇ acyloxy, carboxy, oxycarboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, substituted or unsubstituted, such that, for example, a biphenyl or naphthyl group results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3, or 4-hydroxyphenyl, 2,4-dihydroxyphenyl, the protected-hydroxy derivatives thereof and the like; a nitrophenyl group such as 2, 3, or 4-nitrophenyl; a cyanophenyl group, for example, 2, 3 or 4-cyanophenyl; a mono- or di(alkyl)phenyl group such as 2, 3, or 4-methylphenyl, 2,4-dimethylphenyl, 2, 3 or 4-(iso-propyl)phenyl, 2, 3, or 4-ethylphenyl, 2, 3 or 4-(n-propyl)phenyl and the like; a mono or di(alkoxy)phenyl group, for example, 2,6-dimethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono- or di(hydroxymethyl)phenyl or (protected hydroxymethyl)phenyl such as 2, 3 or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl and the like.

The term “(substituted phenyl)alkyl” means one of the above substituted phenyl groups attached to one of the above-described alkyl groups. Examples of include such groups as 2-phenyl-1-chloroethyl, 2-(4′-methoxyphenyl)ethyl, 4-(2′,6′-dihydroxyphenyl)n-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl and the like.

As noted above, the term “aromatic” or “aryl” refers to six membered carbocyclic rings. Also as noted above, the term “heteroaryl” denotes optionally substituted five-membered or six-membered rings that have 1 to 4 heteroatoms, such as oxygen, sulfur and/or nitrogen atoms, in particular nitrogen, either alone or in conjunction with sulfur or oxygen ring atoms.

Furthermore, the above optionally substituted five-membered or six-membered rings can optionally be fused to an aromatic 5-membered or 6-membered ring system. For example, the rings can be optionally fused to an aromatic 5-membered or 6-membered ring system such as a pyridine or a triazole system, and preferably to a benzene ring.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzthiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C₁ to C₄ alkoxy group, similarly, “lower alkylthio” means a C₁ to C₄ alkylthio group.

The term “(monosubstituted)amino” refers to an amino group with one substituent chosen from the group consisting of phenyl, substituted phenyl, alkyl, substituted alkyl, C₁ to C₄ acyl, C₂ to C₇ alkenyl, C₂ to C₇ substituted alkenyl, C₂ to C₇ alkynyl, C₇ to C₁₆ alkylaryl, C₇ to C₁₆ substituted alkylaryl and heteroaryl group. The (monosubstituted) amino can additionally have an amino-protecting group as encompassed by the term “protected (monosubstituted)amino.” The term “(disubstituted)amino” refers to amino groups with two substituents chosen from the group consisting of phenyl, substituted phenyl, alkyl, substituted alkyl, C₁ to C₇ acyl, C₂ to C₇ alkenyl, C₂ to C₇ alkynyl, C₇ to C₁₆ alkylaryl, C₇ to C₁₆ substituted alkylaryl and heteroaryl. The two substituents can be the same or different.

The term “heteroaryl(alkyl)” denotes an alkyl group as defined above, substituted at any position by a heteroaryl group, as above defined.

“Optional” or “optionally” means that the subsequently described event, circumstance, feature or element may, but need not, occur, and that the description includes instances where the event or circumstance occurs and instances in which it does not. For example, “heterocyclo group optionally mono- or di- substituted with an alkyl group” means that the alkyl may, but need not, be present, and the description includes situations where the heterocyclo group is mono- or disubstituted with an alkyl group and situations where the heterocyclo group is not substituted with the alkyl group.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “an aminopyrimidine compound” includes a plurality of such compounds and reference to “the autoimmune disease” includes reference to one or more autoimmune diseases and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides methods of treating immune disorders. Immune disorders include autoimmune disorders, transplant rejection, and graft-versus-host disease.

The present invention provides methods of treating an autoimmune disorder, the methods generally involving administering to an individual in need thereof an effective amount of an aminopyrimidine compound. The present invention provides methods of reducing the risk that an individual will develop an autoimmune disorder, or will exhibit a symptom of an autoimmune disorder, the methods generally involving administering to an individual in need thereof an effective amount of an aminopyrimidine compound.

The present invention provides methods of reducing the risk of transplant rejection, the methods generally involving administering to an individual in need thereof an effective amount of an aminopyrimidine compound. The present invention further provides methods of increasing or enhancing survival of transplanted organs, tissue, or cells in an individual.

The present invention provides methods of reducing the risk of graft-versus-host disease (GVHD). The present invention provides methods of treating GVHD. The methods generally involve administering to an individual in need thereof an effective amount of an aminopyrimidine compound.

### Methods of Treating Immune Disorders

The present invention provides methods of treating immune disorders, including autoimmune disorders, transplant rejection, and graft-versus-host disease. The methods generally involve administering to an individual in need thereof an effective amount of an aminopyrimidine compound.

### Autoimmune Disorders

In some embodiments, the present invention provides methods of treating an autoimmune disorder in an individual. The present invention provides methods of reducing the risk that an individual will develop an autoimmune disorder, or will exhibit a symptom of an autoimmune disorder. The methods generally involve administering to an individual in need thereof an effective amount of an aminopyrimidine compound.

Individuals in need of treatment using a subject method include individuals who have been diagnosed as having an autoimmune disorder. Individuals in need of treatment with a subject method also include individuals who have not yet been diagnosed as having an autoimmune disorder, but who are at risk of developing an autoimmune disorder.

Autoimmune disorders include autoimmune hemolytic anemia, antiphospholipid syndrome, dermatitis, allergic encephalomyelitis, glomerulonephritis, Goodpasture's Syndrome, Graves' Disease, multiple sclerosis, myasthenia gravis, neuritis, ophthalmia, bullous pemphigoid, pemphigus, acute disseminated encephalomyelitis, polyendocrinopathies, purpura, Reiter's Disease, stiff-Man syndrome, inflammation, Guillain-Barre Syndrome, insulin dependent diabetes mellitus (also referred to as Type 1 diabetes), rheumatoid arthritis, autoimmune inflammatory eye disease, adult respiratory distress syndrome, inflammatory bowel disease, dermatitis, thrombotic thrombocytopenic purpura, Sjögren's syndrome, encephalitis, uveitis, leukocyte adhesion deficiency, psoriatic arthritis, progressive systemic sclerosis, primary biliary cirrhosis, pemphigus, pemphigoid, necrotizing vasculitis, systemic lupus erythematosus, polymyositis, sarcoidosis, granulomatosis, vasculitis, pernicious anemia, CNS inflammatory disorder, antigen-antibody complex mediated diseases, Hashimoto's thyroiditis, habitual spontaneous abortions, Reynard's syndrome, glomerulonephritis, dermatomyositis, chronic active hepatitis, celiac disease, tissue specific autoimmunity, degenerative autoimmunity delayed hypersensitivities, autoimmune complications of acquired immunodeficiency syndrome (AIDS), atrophic gastritis, ankylosing spondylitis and Addison's disease. In some embodiments, one or more of the above-listed autoimmune disorders is specifically excluded. For example, in some embodiments, rheumatoid arthritis is specifically excluded.

In some embodiments, an effective amount of an aminopyrimidine compound is an amount that is effective to reduce the severity of one or more symptoms of an autoimmune disease. For example, in some embodiments, an effective amount of an aminopyrimidine compound is an amount that is effective to reduce the severity of one or more symptoms of an autoimmune disease by at least about 5%, at least about 10%, at least about 25%, at least about 30%, at least about 40%, at least about 50%, at least about 60%, at least about 70%, at least about 80%, or at least about 90%, or more, when compared to the severity of the symptom in an individual not treated with the aminopyrimidine compound.

Symptoms associated with autoimmune disorders are known in the art. See, e.g., “Textbook of the Autoimmune Diseases” R. G. Lahita, Ed. (2000) Lippincott Williams & Wilkins, 1^{st} ed. The following are non-limiting examples.

Multiple sclerosis is characterized by various symptoms and signs of central nervous system (CNS) dysfunction, with remissions and recurring exacerbations. The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances.

Diabetes Mellitus is syndrome characterized by hyperglycemia resulting from absolute or relative impairment in insulin secretion and/or insulin action. Although it may occur at any age, type I DM most commonly develops in childhood or adolescence and is the predominant type of DM diagnosed before age 30. This type of diabetes accounts for 10 to 15% of all cases of DM and is characterized clinically by hyperglycemia.

In some embodiments, a subject method is effective in reducing autoreactivity, where “reducing autoreactivity” includes one or more of reducing the number of autoreactive cells; reducing the activity of an autoreactive cell; and reducing the level of autoreactive antibody. Autoreactivity depends on the interactions of a number of white blood cells, including but not limited to, T lymphocytes, B cells, natural killer (NK) cells and dendritic cells. T lymphocytes include CD4⁺ T lymphocytes and CD8⁺ lymphocytes. B cells can function both as antigen presenting cells and producers of autoantibodies that can target tissues. In some embodiments, the subject method can alter the activities or numbers of these cells involved in various autoimmune reactivities. In some embodiments, a subject method is effective to reduce the number and/or activity of an autoreactive cell in an individual by at least about 5%, at least about 10%, at least about 25%, at least about 30%, at least about 40%, at least about 50%, at least about 60%, at least about 70%, at least about 80%, or at least about 90%, or more, when compared to the number and/or level of autoreactive cells in the individual not treated with the aminopyrimidine compound.

In some embodiments, a subject method is effective to reduce the number and/or activity of an autoreactive T lymphocyte. Thus, in some embodiments, an effective amount of an aminopyrimidine compound is an amount that is effective to reduce the number and/or activity of autoreactive T lymphocytes in an individual by at least about 5%, at least about 10%, at least about 25%, at least about 30%, at least about 40%, at least about 50%, at least about 60%, at least about 70%, at least about 80%, or at least about 90%, or more, when compared to the number and/or level of autoreactive T lymphocytes in the individual not treated with the aminopyrimidine compound.

In some embodiments, a subject method is effective to reduce the number and/or activity of an autoreactive B cell. Thus, in some embodiments, an effective amount of an aminopyrimidine compound is an amount that is effective to reduce the number and/or activity of autoreactive B cells in an individual by at least about 5%, at least about 10%, at least about 25%, at least about 30%, at least about 40%, at least about 50%, at least about 60%, at least about 70%, at least about 80%, or at least about 90%, or more, when compared to the number and/or level of autoreactive B cells in the individual not treated with the aminopyrimidine compound.

Activities of an autoreactive T lymphocyte include, but are not limited to, cytolytic activity toward a “self” cell; secretion of cytokine(s); secretion of chemokine(s); responsiveness to chemokine(s); and trafficking. In some embodiments, an effective amount of an aminopyrimidine compound is an amount that is effective to reduce one or more activities of an autoreactive T lymphocyte in an individual.

Whether an aminopyrimidine compound is effective to reduce the number and/or activity of an autoreactive T lymphocyte in an individual is readily determined using known assays. For example, where the autoreactive T lymphocytes are specific for an autoantigen, the number and activity level of autoantigen-specific T lymphocytes is determined using, e.g., a mixed lymphocyte reaction in which irradiated cells comprising a detectable label in the cytoplasm and displaying the autoantigen are mixed with lymphocytes from the individual. Release of detectable label from the cytoplasm of the autoantigen-displaying cells indicates the presence in the individual of autoreactive lymphocytes. Methods of detecting autoreactive T lymphocytes associated with Type 1 diabetes are known in the art; and any such methods can be used. See, e.g., U.S. Pat. No. 6,022,697 for a discussion of a method of detecting autoreactive T lymphocytes associated with Type 1 diabetes.

### Type 1 Diabetes

In some embodiments, the present invention provides methods for treating Type 1 diabetes in an individual, the method generally involving administering to an individual having Type 1 diabetes an effective amount of an aminopyrimidine compound.

In some embodiments, an effective amount of an aminopyrimidine compound is an amount that is effective to reduce a blood glucose level in an individual by at least about 10%, at least about 15%, at least about 20%, at least about 25%, at least about 30%, at least about 40%, or at least about 50% when compared to the blood glucose levels in the absence of the active agent. In some embodiments, an effective amount of an aminopyrimidine compound is an amount that is effective to reduce blood glucose levels to a normal range. Normal fasting blood glucose levels are typically in the range of from about 70 mg/dL to about 110 mg/dL before a meal. Normal blood glucose levels 2 hours after a meal are usually less than about 120 mg/dL. Normal blood glucose levels during an oral glucose tolerance test (involving drinking a sugar solution containing about 75 g glucose; then measuring blood glucose levels at various times following drinking the sugar solution) include: less than 140 mg/dL 2 hours after drinking the sugar solution; and all readings between 0 and 2 hours after drinking the sugar solution less than 200 mg/dL. Blood glucose levels are also sometimes expressed in mmol/L. Normal blood glucose levels are generally between about 4 mmol/L and 8 mmol/L. Normal blood glucose levels are generally less than about 10 mmol/L 90 minutes after a meal; and from about 4 mmol/L to about 7 mmol/L before meals.

Whether a given aminopyrimidine compound reduces blood glucose levels is readily determined using, e.g., an experimental animal model of Type 1 diabetes. A suitable experimental (non-human) animal model of Type 1 diabetes is the non-obese diabetic mouse.

In some embodiments, an effective amount of an aminopyrimidine compound is an amount that is effective to increase the level of C-peptide to a normal level. Normal levels of C-peptide range from about 0.5 ng/mL to about 3.0 ng/mL for fasting levels of C-peptide.

The non-obese diabetic (NOD) mouse is a model for insulin-dependent diabetes mellitus (IDDM; or Type 1 diabetes), in which the main clinical feature is elevated blood glucose levels (hyperglycemia). The elevated blood glucose level is caused by auto-immune destruction of insulin-producing (3 cells in the islets of Langerhans of the pancreas. Destruction of the β cell is accompanied by a massive cellular infiltration surrounding and penetrating the islets (insulitis) composed of a heterogeneous mixture of CD4⁺ and CD8⁺ T lymphocytes, B lymphocytes, macrophages and dendritic cells. Thus, the NOD mouse represents a model in which auto-immunity against beta-cells is the primary event in the development of IDDM.

In some embodiments, an aminopyrimidine compound is administered following a meal, e.g., within 2 hours after a meal, e.g., from about 1 minute to about 2 hours after a meal. In other embodiments, an active agent is administered before a meal, e.g., from about 1 minute to about 120 minutes before a meal. In other embodiments, an aminopyrimidine compound is administered as needed to lower blood glucose levels, e.g., an active agent is administered within about 1 minute to about 30 minutes following a blood glucose measurement that indicates that the blood glucose level exceeds the normal range. In other embodiments, an aminopyrimidine compound is administered continuously.

In some embodiments, an aminopyrimidine compound is administered to an individual who has been diagnosed with Type I diabetes. In other embodiments, an aminopyrimidine compound is administered to an individual who is at risk of developing Type I diabetes. In some embodiments, an aminopyrimidine compound is administered to an individual who has Type I diabetes, and who is a recipient of a pancreatic islet cell transplant.

In some embodiments, an aminopyrimidine compound is administered to an individual who has been diagnosed with Type I diabetes, and is administered to the individual following diagnosis, where the individual retains at least some islet cell function, e.g., where the individual retains at least some insulin-producing tissue function. For example, in some embodiments, an aminopyrimidine compound is administered to an individual who has Type I diabetes, where the individual retains at least about 5%, at least about 10%, at least about 15%, at least about 20%, at least about 25%, at least about 30%, at least about 40%, at least about 50%, at least about 60%, at least about 70%, or at least about 80%, or more, insulin-producing tissue function based on glucose tolerance testing or other insulin measurements.

In some embodiments, an aminopyrimidine compound is administered to an individual who has been diagnosed as being at risk for Type I diabetes, and is administered before an incidence of hyperglycemia, e.g., before hyperglycemia is detected.

In the treatment of Type 1 diabetes, an aminopyrimidine compound is administered in an amount of from about 10 mg to about 1000 mg per dose, e.g., from about 10 mg to about 20 mg, from about 20 mg to about 25 mg, from about 25 mg to about 50 mg, from about 50 mg to about 75 mg, from about 75 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 750 mg, or from about 750 mg to about 1000 mg per dose.

In some embodiments, the amount of an aminopyrimidine compound per dose is determined on a per body weight basis. For example, in some embodiments, an aminopyrimidine compound is administered in an amount of from about 0.5 mg/kg to about 50 mg/kg, e.g., from about 0.5 mg/kg to about 1 mg/kg, from about 1 mg/kg to about 2 mg/kg, from about 2 mg/kg to about 3 mg/kg, from about 3 mg/kg to about 5 mg/kg, from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, or from about 40 mg/kg to about 50 mg/kg per dose. In other embodiments, an aminopyrimidine compound is administered in an amount of from about 5 mg/kg to about 100 mg/kg, e.g., from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, from about 40 mg/kg to about 50 mg/kg, from about 50 mg/kg to about 60 mg/kg, from about 60 mg/kg to about 70 mg/kg, from about 70 mg/kg to about 80 mg/kg, from about 80 mg/kg to about 90 mg/kg, or from about 90 mg/kg to about 100 mg/kg per dose.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, multiple doses of an aminopyrimidine compound are administered. The frequency of administration of an aminopyrimidine compound can vary depending on any of a variety of factors, e.g., severity of the symptoms, etc. For example, in some embodiments, an aminopyrimidine compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in some embodiments, an aminopyrimidine compound is administered continuously.

The duration of administration of an aminopyrimidine compound, e.g., the period of time over which an aminopyrimidine compound is administered, can vary, depending on any of a variety of factors, e.g., patient response, etc. For example, an aminopyrimidine compound can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, an aminopyrimidine compound is administered for the lifetime of the individual.

In some embodiments, administration of an aminopyrimidine compound is discontinuous, e.g., an aminopyrimidine compound is administered for a first period of time and at a first dosing frequency; administration of the aminopyrimidine compound is suspended for a period of time; then the aminopyrimidine compound is administered for a second period of time for a second dosing frequency. The period of time during which administration of the aminopyrimidine compound is suspended can vary depending on various factors, e.g., blood glucose levels; and will generally range from about 1 week to about 6 months, e.g., from about 1 week to about 2 weeks, from about 2 weeks to about 4 weeks, from about one month to about 2 months, from about 2 months to about 4 months, or from about 4 months to about 6 months, or longer. The first period of time may be the same or different than the second period of time; and the first dosing frequency may be the same or different than the second dosing frequency.

The following are exemplary, non-limiting examples of dosing regimens. In one embodiment, an aminopyrimidine compound is administered orally in an amount of 50 mg/kg daily for a period of 8 weeks. In another embodiment, an aminopyrimidine compound is administered orally in an amount of 50 mg/kg daily for a period of 10 weeks. In another embodiment, an aminopyrimidine compound is administered orally in an amount of 50 mg/kg daily for a period of 2 months. In another embodiment, an aminopyrimidine compound is administered orally in an amount of 50 mg/kg daily for a period of 4 months. In another embodiment, an aminopyrimidine compound is administered orally in an amount of 50 mg/kg daily for a period of 6 months. In another embodiment, an aminopyrimidine compound is administered orally in an amount of 50 mg/kg daily for a period of 12 months or longer.

In one embodiment, an aminopyrimidine compound is administered orally in an amount of 100 mg/kg daily for a period of 8 weeks. In another embodiment, an aminopyrimidine compound is administered orally in an amount of 100 mg/kg daily for a period of 10 weeks. In another embodiment, an aminopyrimidine compound is administered orally in an amount of 100 mg/kg daily for a period of 2 months. In another embodiment, an aminopyrimidine compound is administered orally in an amount of 100 mg/kg daily for a period of 4 months. In another embodiment, an aminopyrimidine compound is administered orally in an amount of 100 mg/kg daily for a period of 6 months. In another embodiment, an aminopyrimidine compound is administered orally in an amount of 100 mg/kg daily for a period of 12 months or longer.

In one embodiment, an aminopyrimidine compound is administered orally in an amount of 50 mg/kg three times per week for a period of 8 weeks. In another embodiment, an aminopyrimidine compound is administered orally in an amount of 50 mg/kg three times per week for a period of 10 weeks. In another embodiment, an aminopyrimidine compound is administered orally in an amount of 50 mg/kg three times per week for a period of 2 months. In another embodiment, an aminopyrimidine compound is administered orally in an amount of 50 mg/kg three times per week for a period of 4 months. In another embodiment, an aminopyrimidine compound is administered orally in an amount of 50 mg/kg three times per week for a period of 6 months. In another embodiment, an aminopyrimidine compound is administered orally in an amount of 50 mg/kg three times per week for a period of 12 months or longer.

In one embodiment, an aminopyrimidine compound is administered orally in an amount of 100 mg/kg three times per week for a period of 8 weeks. In another embodiment, an aminopyrimidine compound is administered orally in an amount of 100 mg/kg three times per week for a period of 10 weeks. In another embodiment, an aminopyrimidine compound is administered orally in an amount of 100 mg/kg three times per week for a period of 2 months. In another embodiment, an aminopyrimidine compound is administered orally in an amount of 100 mg/kg three times per week for a period of 4 months. In another embodiment, an aminopyrimidine compound is administered orally in an amount of 100 mg/kg three times per week for a period of 6 months. In another embodiment, an aminopyrimidine compound is administered orally in an amount of 100 mg/kg three times per week for a period of 12 months, or longer.

In another embodiment, a pre-diabetic individual is treated with an aminopyrimidine compound. Pre-diabetic individuals include individuals who are at risk of developing Type 1 diabetes. Parameters associated with increased risk of developing Type 1 diabetes are known in the art; see, e.g., Diabetes Prevention Trial—Type 1 diabetes study group (2002) N. Engl. J. Med. 346:1685-1691 for examples of such parameters. Examples of individuals at risk of developing Type I diabetes include individuals having one or more of the following: circulating antibodies specific for islet cells (e.g., individuals with titers of 10 JDF units or higher); an HLA haplotype associated with increased risk of developing Type 1 diabetes; and a family history of Type 1 diabetes.

### Methods of Reducing the Risk of Transplant Rejection

In some embodiments, the present invention provides methods for reducing the risk of transplant rejection, e.g., methods for increasing transplant survival in an individual. The methods generally involve administering to an individual in need thereof an effective amount of an aminopyrimidine compound. Transplants include organs, tissues, and cells.

In some embodiments, an effective amount of an aminopyrimidine compound is an amount that is effective to increase the survival of the transplanted organ, tissue, or cells. Thus, in some embodiments, an effective amount of an aminopyrimidine compound is an amount that is effective to increase the survival of transplanted organ, tissue, or cells in a transplant recipient by at least about 5%, at least about 10%, at least about 15%, at least about 20%, at least about 30%, at least about 40%, at least about 50%, at least about 100% or 2-fold, at least about 5-fold, at least about 10-fold, or more, compared to the survival of the transplanted organ, tissue or cells in a transplant recipient not treated with the aminopyrimidine compound. “Increasing the survival” of a transplanted organ, tissue, or cells refers to increasing one or more of: a) the time period that a transplanted organ, tissue, or cells remains in the transplant recipient without being rejected; and b) one or more functions of a transplanted organ, tissue, or cells in the transplant recipient. Functions of transplanted organ, tissue, or cells depend on the particular organ, tissue, or cells. For example, a function of transplanted pancreatic islet tissue is production and secretion of insulin.

In some embodiments, an effective amount of an aminopyrimidine compound is an amount that is effective to decrease the number and/or activity of alloreactive cells, or other cells that participate in an alloimmune response, in the transplant recipient. Alloreactive cells include, but are not limited to, T lymphocytes (e.g., CD4⁺ T cells, CD8⁺ T cells), and B cells. Other cells that are not defined as alloreactive, but that participate in an alloimmune response include natural killer (NK) cells and dendritic cells (DC). Thus, in some embodiments, an effective amount of an aminopyrimidine compound is an amount that is effective to reduce the number and/or activity of alloreactive cells, or other cells that participate in an alloimmune response, in a transplant recipient by at least about 5%, at least about 10%, at least about 15%, at least about 20%, at least about 30%, at least about 40%, at least about 50%, at least about 60%, at least about 70%, at least about 80%, at least about 90%, or more, compared to the number and/or activity of alloreactive cells, or other cells that participate in an alloimmune response, in the transplant recipient not treated with the aminopyrimidine compound.

In some embodiments, an effective amount of an aminopyrimidine compound is an amount that is effective to decrease the number and/or activity of alloreactive T lymphocytes in the transplant recipient. Thus, in some embodiments, an effective amount of an aminopyrimidine compound is an amount that is effective to reduce the number and/or activity of alloreactive T lymphocytes in a transplant recipient by at least about 5%, at least about 10%, at least about 15%, at least about 20%, at least about 30%, at least about 40%, at least about 50%, at least about 60%, at least about 70%, at least about 80%, at least about 90%, or more, compared to the number and/or activity of alloreactive T lymphocytes in the transplant recipient not treated with the aminopyrimidine compound.

The transplant recipient is typically a mammal. The organ, tissue, or cells to be transplanted into the recipient will in some embodiments be from the same species as the recipient, e.g., the organ, tissue, or cells to be transplanted is/are an allograft. In other embodiments, organ, tissue, or cells to be transplanted into the recipient will be from another species, e.g., the organ, tissue, or cells to be transplanted is/are a xenograft.

Tissues or organs which may be transplanted include, but are not limited to, heart, liver, kidney, lung, pancreas, pancreatic islets, brain tissue, cornea, bone, intestine, and skin. Cells that may be transplanted include, but are not limited to, lymphocytes, dopamine-producing cells, bone marrow cells, stem cells, and blood cells. In some embodiments, a selected subset of cells is transplanted, e.g., a sub-population of cells selected for antigen specificity, display of one or more cell-surface antigens, cytokine secretion profile, etc., is transplanted. In addition, the cells, cell population, or cell sub-population will in some embodiments be treated before transplanting into the recipient.

A number of different cell types have been considered for therapeutic purposes, including somatic cells as diverse as hematopoietic stem cells; mesenchymal stem cells; and peripheral blood cells. Included in cells for therapy are cells derived from embryonic stem cells (ES cells). ES cells have the capacity to give rise to all tissues, including those for which no somatic stem cells are known, such as cardiac muscle (see Kehat et al. (2001) J. Clin. Invest. 108:407-414; Mummery et al. (2002) J. Anat. 200:233-242). ES cells have certain advantages for cardiac repair applications. There are well-defined protocols for the isolation and maintenance of ESCs, and they have a tremendous capacity for in vitro expansion, making them scalable for human applications (Zandstra et al. (2003) Tissue Eng. 9:767-778).

In some embodiments, an aminopyrimidine compound is administered before an organ, tissue, or cells is/are transplanted into a recipient, e.g., an aminopyrimidine compound is administered to a prospective transplant recipient. In these embodiments, an aminopyrimidine compound is administered to the prospective transplant recipient at least about 5 minutes, at least about 30 minutes, at least about 1 hour, at least about 4 hours, at least about 8 hours, at least about 12 hours, at least about 24 hours, at least about 48 hours, or at least about 72 hours, or longer, before the organ, tissue, or cells is/are transplanted into the recipient.

In some embodiments, an aminopyrimidine compound is administered to the transplant recipient (e.g., an individual who has received an organ, tissue, or cells) immediately following the transplant and for a period of time of from about one week to one month, from about one month to about 6 months, from about 6 months to about one year, or more than one year, following transplantation of the organ, tissue, or cells into the recipient.

In some embodiments, multiple doses of an aminopyrimidine compound are administered. The frequency of administration of an aminopyrimidine compound can vary depending on any of a variety of factors. For example, in some embodiments, an aminopyrimidine compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in some embodiments, an aminopyrimidine compound is administered continuously.

Whether a given aminopyrimidine compound is effective to increase transplant survival is readily determined. A number of biochemical markers may be used to detect allograft rejection. A number of these are nonspecific indicators of inflammation, e.g., white blood cell count (WBC), while a number of others are specific to the transplanted organ. For example, during liver rejection, plasma levels of liver enzymes such as aspartate transaminase (AST) and alanine aminotransferase (ALT) may be elevated. As another example, researchers have found cardiac transplant patients were at greater risk of developing coronary artery disease (the leading cause of transplant failure) when they tested positive within three months after transplant for the presence of two specific marker molecules, ICAM-1 and HLA-DR, in the inner lining of the coronary arteries. The researchers found the marker molecules during routine endomyocardial biopsy specimens performed to monitor the patients for transplant rejection. This is a potential early warning sign to physicians, as patients who developed the marker molecules in the inner lining of the coronary arteries were four times more likely than those who did not to experience transplant rejection years down the road. Lung transplant patients are monitored for rejection by one or more of X-ray or other imaging methods, pulmonary function tests, transbronchial biopsy histology, and bronchoalveolar lavage analysis.

In addition, a biopsy of the transplanted organ, tissue, or cells may be analyzed in any of a number of ways to assess the survival of the transplanted organ, tissue, or cells. A biopsy may be analyzed histologically for the presence of cell surface markers; cellular infiltrates; cellular damage; etc. A biopsy may be analyzed for functional characteristics. Such analytical methods are known to those skilled in the art. For example, histopathological examination of a renal biopsy may enable a differential diagnosis between rejection and cyclosporine toxicity. Immunostaining of renal tubular cells, a primary target of infiltrating T cells, shows increased expression of HLA class II antigens during rejection. Heart transplant patients are monitored by histopathological analysis of endomyocardial biopsies at regular intervals. These biopsies are obtained through a catheter passed into the right ventricle; histological rejection is assessed by the degree of cellular infiltration and myocyte damage.

### Methods of Treating Graft-Versus-Host Disease

In some embodiments, the present invention provides methods for reducing the risk of graft-versus-host disease (GVHD) in an individual; methods of reducing the risk of death due to GVHD; and methods of reducing the severity of GVHD. The methods generally involve administering to an individual in need thereof an effective amount of an aminopyrimidine compound.

GVHD typically involves the donor cells attacking the host (transplant recipient). For example, bone marrow transplantation (BMT) is used in conjunction with treatments of a number of cancers, particularly treatments that damage or destroy cell types found in blood, such as treatments of life-threatening hematologic malignancies. However, the threat of severe graft-vs.-host disease (GVHD) remains a major obstacle, impeding widespread application of bone marrow transplantation. Acute and chronic GVHD develops in a significant proportion of transplant recipients and represents a major cause of morbidity and mortality after bone marrow transplantation between imperfectly matched individuals (i.e., allogeneic transplantation). Efforts to prevent GVHD should reduce acute toxicity and morbidity of transplantation, and also to enhance the long term outcome of a transplant. GVHD is a T-cell mediated disease affecting multiple organ systems.

In some embodiments, an effective amount of an aminopyrimidine compound is an amount that is effective to decrease the risk of GVH disease by at least about 5%, at least about 10%, at least about 15%, at least about 20%, at least about 30%, at least about 40%, at least about 50%, at least about 60%, at least about 70%, at least about 80%, at least about 90%, or more, compared with the risk of GVHD in the individual (graft recipient) not treated with the aminopyrimidine compound.

In some embodiments, an effective amount of an aminopyrimidine compound is an amount that is effective to decrease the risk of death by GVH disease by at least about 5%, at least about 10%, at least about 15%, at least about 20%, at least about 30%, at least about 40%, at least about 50%, at least about 60%, at least about 70%, at least about 80%, at least about 90%, or more, compared with the risk of death by GVHD in the individual (graft recipient) not treated with the aminopyrimidine compound.

In some embodiments, an effective amount of an aminopyrimidine compound is an amount that is effective to increase the survival of the transplanted organ, tissue, or cells. Thus, in some embodiments, an effective amount of an aminopyrimidine compound is an amount that is effective to increase the survival of transplanted organ, tissue, or cells in a transplant recipient by at least about 5%, at least about 10%, at least about 15%, at least about 20%, at least about 30%, at least about 40%, at least about 50%, at least about 100% or 2-fold, at least about 5-fold, at least about 10-fold, or more, compared to the survival of the transplanted organ, tissue or cells in a transplant recipient not treated with the aminopyrimidine compound.

In some embodiments, an effective amount of an aminopyrimidine compound is an amount that is effective to decrease the number and/or activity of alloreactive T cells (e.g., cells reactive toward the recipient) or other white blood cells involved in alloimmune responses including B cells, NK cells and dendritic cells that may be present in the transplant organ, tissue, or cells (also referred to as “graft organ, tissue, or cells”) or in draining lymphoid organs. Infiltrating cells that may be present in the graft organ, tissue, or cells include T lymphocytes, natural killer (NK) cells, B cells, dendritic cells, etc. Thus, in some embodiments, an effective amount of an aminopyrimidine compound is an amount that is effective to reduce the number and/or activity of alloreactive T cells or other white blood cells involved in alloimmune responses including B cells, NK cells and dendritic cells in the transplant organ, tissue, or cells or in draining lymphoid organs by at least about 5%, at least about 10%, at least about 15%, at least about 20%, at least about 30%, at least about 40%, at least about 50%, at least about 60%, at least about 70%, at least about 80%, at least about 90%, or more, compared to the number and/or activity of alloreactive cells present in the transplant organ, tissue, or cells when the recipient is not treated with the aminopyrimidine compound.

In some embodiments, an effective amount of an aminopyrimidine compound is an amount that is effective to decrease the number and/or activity of alloreactive T lymphocytes (e.g., T lymphocytes reactive toward the recipient) that may be present in the graft organ, tissue, or cells. Thus, in some embodiments, an effective amount of an aminopyrimidine compound is an amount that is effective to reduce the number and/or activity of alloreactive T lymphocytes in the graft organ, tissue, or cells or in draining lymphoid organs by at least about 5%, at least about 10%, at least about 15%, at least about 20%, at least about 30%, at least about 40%, at least about 50%, at least about 60%, at least about 70%, at least about 80%, at least about 90%, or more, compared to the number and/or activity of alloreactive T lymphocytes present in the graft organ, tissue, or cells when the recipient is not treated with the aminopyrimidine compound.

In some embodiments, an effective amount of an aminopyrimidine compound is an amount that is effective to decrease the number and/or activity of NK cells that may be present in the graft organ, tissue, or cells or in draining lymphoid organs. Thus, in some embodiments, an effective amount of an aminopyrimidine compound is an amount that is effective to reduce the number and/or activity of NK cells in the graft organ, tissue, or cells by at least about 5%, at least about 10%, at least about 15%, at least about 20%, at least about 30%, at least about 40%, at least about 50%, at least about 60%, at least about 70%, at least about 80%, at least about 90%, or more, compared to the number and/or activity of alloreactive NK cells present in the graft organ, tissue, or cells when the recipient is not treated with the aminopyrimidine compound.

In some embodiments, an aminopyrimidine compound is administered to the transplant recipient (e.g., an individual who has received an organ, tissue, or cells) immediately following the transplant and for a period of time of from about one week to one month, from about one month to about 6 months, from about 6 months to about one year, or more than one year, following transplantation of the organ, tissue, or cells into the recipient.

In some embodiments, multiple doses of an aminopyrimidine compound are administered. The frequency of administration of an aminopyrimidine compound can vary depending on any of a variety of factors. For example, in some embodiments, an aminopyrimidine compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (lid). As discussed above, in some embodiments, an aminopyrimidine compound is administered continuously.

### Aminopyrimidine Compounds

Aminopyrimidine compounds suitable for use in a subject method include aminopyrimidine compounds that inhibit one or more tyrosine kinases, e.g., one or more of ABL, Src, PDGF-R, and kit. In many embodiments, an aminopyrimidine compound that is suitable for use in a subject method is an aminopyrimidine compound that inhibits one or more tyrosine kinases with an IC₅₀ of less than about 500 nM, e.g., the aminopyrimidine compound inhibits one or more tyrosine kinases with an IC₅₀ of from about 500 nM to about 400 nM, from about 400 nM to about 300 nM, from about 300 nM to about 250 nM, from about 250 nM to about 200 nM, from about 200 nM to about 150 nM, from about 150 nM to about 100 nM, from about 100 nM to about 50 nM, from about 50 nM to about 30 nM, from about 30 nM to about 25 nM, from about 25 nM to about 20 nM, from about 20 nM to about 15 nM, from about 15 nM to about 10 nM, from about 10 nM to about 5 nM, or less than about 5 nM.

Aminopyrimidine compounds suitable for use in a subject method include a compound of any of Formulas I-XI, as shown below; as well as derivatives, prodrugs, analogs, and pharmaceutically acceptable salt thereof.

Aminopyrimidine compounds suitable for use in a subject method include compounds of Formula Ia:

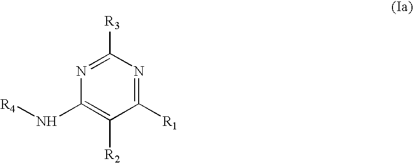

where R₁, R₂, R₃, and R₄ are each independently H, OH, N, NH, lower alkyl, or an aliphatic group having at least 5 carbon atoms, or an aromatic, aromatic-aliphatic, cycloaliphatic, cycloaliphatic-aliphatic, substituted or unsubstituted heterocyclic or heterocyclic-aliphatic group, or as described further below.

Aminopyrimidine compounds suitable for use in a subject method include compounds of Formula Ib:

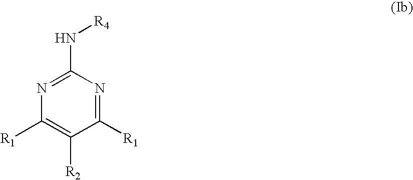

where R₁, R₂, R₃, and R₄ are each independently H, OH, N, NH, lower alkyl, or an aliphatic group having at least 5 carbon atoms, or an aromatic, aromatic-aliphatic, cycloaliphatic, cycloaliphatic-aliphatic, substituted or unsubstituted heterocyclic or heterocyclic-aliphatic group, or as described further below.

In some embodiments, aminopyrimidine compounds suitable for use in a subject method are N-phenyl-2-pyrimidine-amine derivatives of formula (Ic):

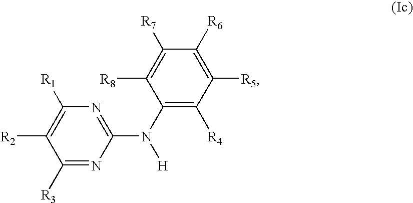

wherein

R₁ is 4-pyrazinyl, 1-methyl-1H-pyrrolyl, amino- or amino-lower alkyl-substituted phenyl wherein the amino group in each case is free, alkylated or acylated, 1H-indolyl or 1H-imidazolyl bonded at a five-membered ring carbon atom, or unsubstituted or lower alkyl-substituted pyridyl bonded at a ring carbon atom and unsubstituted or substituted at the nitrogen atom by oxygen,
R₂ and R₃ are each independently of the other hydrogen, or lower alkyl;
one or two of R₄, R₅, R₆, R₇, and R₈ are each independently nitro, fluoro-substituted lower alkoxy or a radical of formula (II):

—N(R₉)—C(═X)—(Y)ₖ—R₁₀ (II)

wherein

R₉ is hydrogen or lower alkyl,

X is oxo, thio, imino, N-lower alkyl-imino, hydroximino or O-lower alkyl-hydroximino,

Y is oxygen or the group NH,

k is 0 or 1 and

R₁₀ is an aliphatic group having at least 5 carbon atoms, or an aromatic, aromatic-aliphatic, cycloaliphatic, cycloaliphatic-aliphatic, heterocyclic or heterocyclic-aliphatic group,

and the remaining groups R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterified hydroxy, free, alkylated or acylated amino or free or esterified carboxy,

and salts of such compounds having at least one salt-forming group.

For example, in some embodiments, one of R₄, R₅, R₆, R₇, and R₈ is of the formula III:

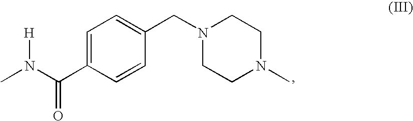

and the remaining groups R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterified hydroxy, free, alkylated or acylated amino or free or esterified carboxy.

Alternatively, in some embodiments, R₂ and R₃ are each independently of the other hydrogen, lower alkyl,
one or two of R₄, R₅, R₆, R₇, and R₈ are each independently nitro, fluoro-substituted lower alkoxy or a radical of formula (IV):

—C(═X)—N(R₉)—(Y)ₖ—R₁₀ (IV)

wherein

R₉ is hydrogen or lower alkyl,

X is oxo, thio, imino, N-lower alkyl-imino, hydroximino or O-lower alkyl-hydroximino,

Y is oxygen or the group NH,

k is 0 or 1 and

R₁₀ is an aliphatic group having at least 5 carbon atoms, or an aromatic, aromatic-aliphatic, cycloaliphatic, cycloaliphatic-aliphatic, heterocyclic or heterocyclic-aliphatic group,

and the remaining groups R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterified hydroxy, free, alkylated or acylated amino or free or esterified carboxy,

and salts of such compounds having at least one salt-forming group.

For example, in some embodiments, one of R₄, R₅, R₆, R₇, and R₈ is of the formula V:

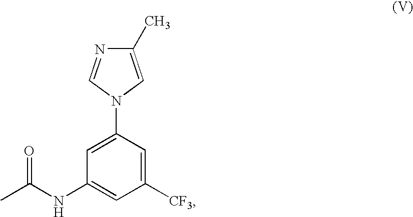

and the remaining groups R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterified hydroxy, free, alkylated or acylated amino or free or esterified carboxy.

In Formula Ic, where R₁ is 1-Methyl-1H-pyrrolyl, in some embodiments, the 1-Methyl-1H-pyrrolyl is 1-methyl-1H-pyrrol-2-yl. Where R₁ is 1-Methyl-1H-pyrrolyl, in some embodiments, then 1-Methyl-1H-pyrrolyl is 1-methyl-1H-pyrrol-3-yl.

In Formula Ic, amino- or amino-lower alkyl-substituted phenyl R₁ wherein the amino group in each case is free, alkylated or acylated, is phenyl substituted in any desired position (ortho, meta or para) wherein an alkylated amino group is preferably mono- or di-lower alkylamino, for example dimethylamino, and the lower alkyl moiety of amino-lower alkyl is preferably linear C₁-C₃ alkyl, such as especially methyl or ethyl.

In Formula Ic, 1H-Indolyl bonded at a carbon atom of the five-membered ring is in some embodiments 1H-indol-2-yl or 1H-indol-3-yl.

In Formula Ic, unsubstituted or lower alkyl-substituted pyridyl bonded at a ring carbon atom is in some embodiments lower alkyl-substituted or unsubstituted 2-, or 3- or 4-pyridyl, for example 3-pyridyl, 2-methyl-3-pyridyl, 4-methyl-3-pyridyl or 4-pyridyl. Pyridyl substituted at the nitrogen atom by oxygen is in some embodiments a radical derived from pyridine N-oxide, i.e., N-oxido-pyridyl, e.g. N-oxido-4-pyridyl.

In Formula Ic, fluoro-substituted lower alkoxy is in some embodiments lower alkoxy carrying at least one, but preferably several, fluoro substituents, e.g., trifluoromethoxy or 1,1,2,2-tetrafluoro-ethoxy.

In Formula Ic, when X is oxo, thio, imino, N-lower alkyl-imino, hydroximino or O-lower alkyl-hydroximino, the group C═X is, in the above order, a radical C═O, C═S, C═N—H, C═N-lower alkyl, C═N—OH or CN—O-lower alkyl, respectively; and X is in some embodiments oxo;

k is in some embodiments 0, i.e., the group Y is not present;

Y, if present, is in some embodiments the group NH;

The term “lower” within the scope of this text denotes radicals having up to and including 7, e.g., up to and including 4 carbon atoms.

In Formula Ic, lower alkyl R₁, R₂, R₃ and R₉ is in some embodiments methyl or ethyl.

In Formula Ic an aliphatic radical R₁₀ having at least 5 carbon atoms generally has not more than 22 carbon atoms, generally not more than 10 carbon atoms, and is such a substituted or unsubstituted aliphatic hydrocarbon radical, that is to say such a substituted or unsubstituted alkynyl, alkenyl or alkyl radical, such as C₅-C₇ alkyl, for example n-pentyl. An aromatic radical R₁₀ has up to 20 carbon atoms and is unsubstituted or substituted, for example in each case unsubstituted or substituted naphthyl, such as especially 2-naphthyl, or phenyl, the substituents being selected from cyano, unsubstituted or hydroxy-, amino- or 4-methyl-piperazinyl-substituted lower alkyl, such as especially methyl, trifluoromethyl, free, etherified or esterified hydroxy, free, alkylated or acylated amino and free or esterified carboxy. In an aromatic-aliphatic radical R₁₀ the aromatic moiety is as defined above and the aliphatic moiety is in some embodiments lower alkyl, such as especially C₁-C₂ alkyl, which is substituted or unsubstituted, for example benzyl. A cycloaliphatic radical R₁₀ has, e.g., up to 30, up to 20, or up to 10 carbon atoms, is mono- or poly-cyclic and is substituted or unsubstituted, for example such a cycloalkyl radical, especially such a 5- or 6-membered cycloalkyl radical, such as cyclohexyl. In a cycloaliphatic-aliphatic radical R₁₀ the cycloaliphatic moiety is as defined above and the aliphatic moiety is in some embodiments lower alkyl, such as especially C₁-C₂ alkyl, which is substituted or unsubstituted. A heterocyclic radical R₁₀ contains especially up to 20 carbon atoms and is preferably a saturated or unsaturated monocyclic radical having 5 or 6 ring members and 1-3 hetero atoms which are preferably selected from nitrogen, oxygen and sulfur, especially, for example, thienyl or 2-, 3- or 4-pyridyl, or a bi- or tri-cyclic radical wherein, for example, one or two benzene radicals are annellated (fused) to the mentioned monocyclic radical. In a heterocyclic-aliphatic radical R₁₀ the heterocyclic moiety is as defined above and the aliphatic moiety is in some embodiments lower alkyl, such as especially C₁-C₂ alkyl, which is substituted or unsubstituted.

Etherified hydroxy is in some embodiments lower alkoxy. Esterified hydroxy is in some embodiments hydroxy esterified by an organic carboxylic acid, such as a lower alkanoic acid, or a mineral acid, such as a hydrohalic acid, for example lower alkanoyloxy or halogen, such as iodine, bromine, fluorine, or chlorine.

Alkylated amino is, for example, lower alkylamino, such as methylamino, or di-lower alkylamino, such as dimethylamino. Acylated amino is, for example, lower alkanoylamino or benzoylamino.

Esterified carboxy is, for example, lower alkoxycarbonyl, such as methoxycarbonyl.

A substituted phenyl radical may carry up to 5 substituents, such as fluorine, but especially in the case of relatively large substituents is generally substituted by only from 1 to 3 substituents. Examples of substituted phenyl that may be given special mention are 4-chloro-phenyl, pentafluoro-phenyl, 2-carboxy-phenyl, 2-methoxy-phenyl, 4-fluorophenyl, 4-cyano-phenyl and 4-methyl-phenyl.

Salt-forming groups in a compound of formula (Ic) are groups or radicals having basic or acidic properties. Compounds having at least one basic group or at least one basic radical, for example a free amino group, a pyrazinyl radical or a pyridyl radical, may form acid addition salts, for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromatic-aliphatic carboxylic acids, such as mandelic acid or cinnamic acid, heterbaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. When several basic groups are present mono- or poly-acid addition salts may be formed.

Compounds of formula (Ic) having acidic groups, for example a free carboxy group in the radical R₁₀, may form metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri-(2-hydroxyethyl)-amine, or heterocyclic bases, for example N-ethylpiperidine or N,N′-dimethyl-piperazine.

Compounds of formula (Ic) having both acidic and basic groups can form internal salts.

Of particular interest in some embodiments is a pyrimidine derivative in which R₁ is 3-pyridyl, R₂, R₃, R₅, R₆, and R₈ are each hydrogen, R_{4′} is methyl, and R₇ is a group of formula (IV) in which R_{9′} is hydrogen, X is oxo, k is 0, and R₁₀ is 4-[(4-methyl-1-piperazinyl)methyl]phenyl. The mesylate salt of this compound having the chemical name 4-[(4-methyl-1-piperazinyl)methyl]-N-[4-methyl-3-[[4-(3-pyridinyl)-2-pyrimidinyl]amino-phenyl]benzamide methanesulfonate is now commonly known as imatinib mesylate and sold under the trademark Gleevec™.

Of particular interest in some embodiments is a phenylaminopyrimidine compound of the formula (VI):

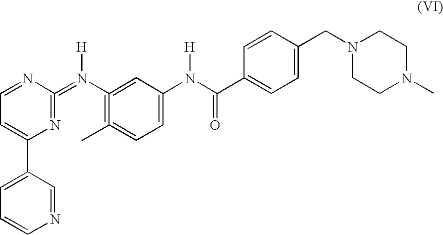

or a pharmaceutically acceptable salt thereof.

Aminopyrimidine compounds suitable for use in a subject method include imatinib mesylate (Formula VI) and derivatives and analogs thereof. Gleevec™ (also known as STI-571; CGP57148B; and imatinib mesylate) has the chemical name 4-[(4-methyl-1-piperazinyl)methyl]-N-[4-methyl-3-[[4-(3-pyridinyl)-2-pyrimidinyl]amino-phenyl]benzamide methanesulfonate is commonly known as imatinib mesylate and sold under the trademark Gleevec™. Gleevec™ is a 2-phenylaminopyrimidine that targets the ATP-binding site of the kinase domain of Bcr-Abl tyrosine kinase (see, e.g. Druker et al. (1996) Nature Med. 2, 561; and Buchdunger et al. (1993) Proc. Natl. Acad. Sci. USA 92:2558-2562).

In certain embodiments, an aminopyrimidine compound suitable for use in a subject method is a compound as described in U.S. Pat. No. 5,521,184. In other embodiments, an aminopyrimidine compound is a compound as described in U.S. Pat. No. 6,958,335.

Of particular interest in some embodiments is a phenylaminopyrimidine compound of the formula (VII):

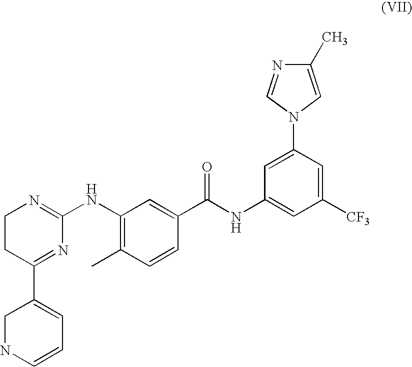

See, e.g., O'Hare et al. (2005) Cancer Res. 65:4500-4505.

In some embodiments, a suitable aminopyrimidine compound is a compound of Formula VIII:

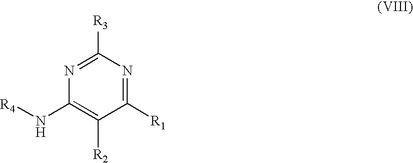

where R₁, R₂, and R₃ are each independently H, N, NH, hydroxyl, lower alkyl, or an aliphatic group having at least 5 carbon atoms, or an aromatic, aromatic-aliphatic, cycloaliphatic, cycloaliphatic-aliphatic, heterocyclic or heterocyclic-aliphatic group;

R₄ is an aliphatic group having at least 5 carbon atoms, or an aromatic, aromatic-aliphatic, cycloaliphatic, cycloaliphatic-aliphatic, heterocyclic or heterocyclic-aliphatic group.

In some embodiments, R₁ and R₂ are a fused, heterocyclic, substituted or unsubstituted cyclopentyl group. In some of these embodiments, R₁ and R₂ are each N, where one or both of R₁ and R₂ are in some embodiments substituted with a lower alkyl, or an aliphatic group having at least 5 carbon atoms, or an aromatic, aromatic-aliphatic, cycloaliphatic, cycloaliphatic-aliphatic, heterocyclic or heterocyclic-aliphatic group.

Thus, in some embodiments, an aminopyrimidine compound is of the formula VIIIb:

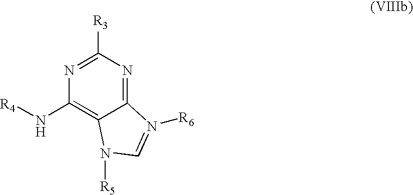

where R₃ is H, lower alkyl, or an aliphatic group having at least 5 carbon atoms, or an aromatic, aromatic-aliphatic, cycloaliphatic, cycloaliphatic-aliphatic, heterocyclic or heterocyclic-aliphatic group;

R₄ is an aliphatic group having at least 5 carbon atoms, or an aromatic, aromatic-aliphatic, cycloaliphatic, cycloaliphatic-aliphatic, heterocyclic or heterocyclic-aliphatic group; and

R₅ and R₆ are each independently H, lower alkyl, or an aliphatic group having at least 5 carbon atoms, or an aromatic, aromatic-aliphatic, cycloaliphatic, cycloaliphatic-aliphatic, heterocyclic or heterocyclic-aliphatic group.

In some embodiments, where the aminopyrimidine compound is a compound of Formula VIIIb:

R₅ is

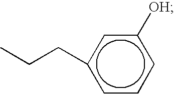

R₆ is H,

R₃ is a substituted or unsubstituted linear or cyclic alkyl group, e.g., a cyclopentyl group; and

R₄ is a substituted or unsubstituted phenyl group, e.g., a phenyl group, e.g., a 3-hydroxyphenylethyl group.

In some embodiments, where the aminopyrimidine compound is of Formula VIII, R₄ is of the formula IX:

—X—C(═Y)—N(R₅)—(Y)ₖ—R₆ (IX)

wherein

X is an aliphatic group having at least 5 carbon atoms, or an aromatic, aromatic-aliphatic, cycloaliphatic, cycloaliphatic-aliphatic, heterocyclic or heterocyclic-aliphatic group, which groups are substituted or unsubstituted;

R₅ is hydrogen or lower alkyl,

Y is oxo, thio, imino, N-lower alkyl-imino, hydroximino or O-lower alkyl-hydroximino,

X is oxygen or the group NH,

k is 0 or 1 and

R₆ is an aliphatic group having at least 5 carbon atoms, or an aromatic, aromatic-aliphatic, cycloaliphatic, cycloaliphatic-aliphatic, heterocyclic or heterocyclic-aliphatic group, which groups are substituted or unsubstituted.

In some embodiments, R₆ is a substituted phenyl group, e.g., substituted with one or more lower alkyl groups and/or one or more halogen moieties.

Thus, in some embodiments, an aminopyrimidine compound is of formula VIIIc:

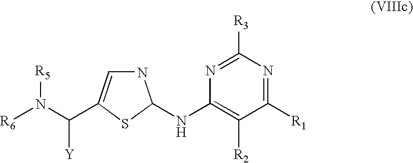

where R₃ is H, lower alkyl, or an aliphatic group having at least 5 carbon atoms, or an aromatic, aromatic-aliphatic, cycloaliphatic, cycloaliphatic-aliphatic, heterocyclic or heterocyclic-aliphatic group;

R₁ is a substituted or unsubstituted heterocyclic group;

R₂ is H or lower alkyl;

R₅ is hydrogen or lower alkyl,

R₆ is an aliphatic group having at least 5 carbon atoms, or an aromatic, aromatic-aliphatic, cycloaliphatic, cycloaliphatic-aliphatic, heterocyclic or heterocyclic-aliphatic group, which groups are substituted or unsubstituted.

In some embodiments of Formula VIIc, R₆ is:

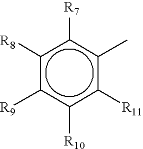

where each of R₇, R₈, R₉, R₁₀, and R₁₁ is independently H, a halo group, a lower alkyl (e.g., methyl, etc.), a hydroxyl, or an aromatic, aromatic-aliphatic, cycloaliphatic, cycloaliphatic-aliphatic, heterocyclic or heterocyclic-aliphatic group, which groups are substituted or unsubstituted.

In some embodiments of Formula VIIc, R₁ is:

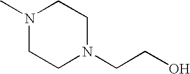

In particular embodiments, a suitable aminopyrimidine compound is N-(2-chloro-6-methyl-phenyl)-2-(6-(4-(2-hydroxyethyl)-piperazin-1-yl)-2-methylpyrimidin-4-ylamino)thiazole-5-carboxamide (BMS-354825; also referred to as Dasatinib); and derivatives and analogs thereof. See, e.g., Lombardo et al. (2004) J. Med. Chem. 47:6658-6661; and Shah et al. (2004) Science 305:399-401. BMS-354825 has the structure of Formula X:

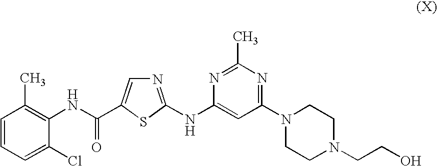

In some embodiments, a suitable aminopyrimidine compound is a compound of Formula XI:

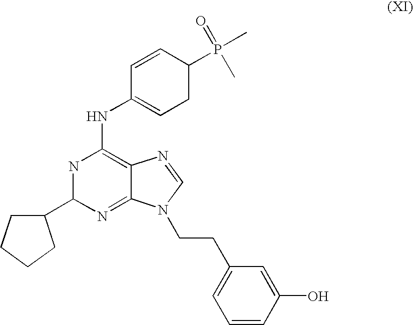

See, e.g., O'Hare et al. (2004) Blood 104:2532-2539.

### Formulations, Dosages, and Routes of Administration

An aminopyrimidine compound (referred generically below as an “active agent”; or “drug”) is formulated with one or more pharmaceutically acceptable excipients. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public

In the subject methods, an aminopyrimidine compound may be administered to the host using any convenient means capable of resulting in the desired reduction in autoimmune disease. Thus, the aminopyrimidine compound can be incorporated into a variety of formulations for therapeutic administration. More particularly, a aminopyrimidine compound can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, an active agent may be administered in the form of their pharmaceutically acceptable salts, or an active agent may be used alone or in appropriate association, as well as in combination, with other pharmaceutically active compounds. The following methods and excipients are merely exemplary and are in no way limiting.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise an active agent in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for an active agent depend on the particular compound employed and the effect to be achieved, and the pharmacodynamics associated with each compound in the host.

An active agent can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

In some embodiments, an active agent is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of active agent can be accomplished using any of a variety of refillable, pump systems. Pumps provide consistent, controlled release over time. In some embodiments, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are used in some embodiments because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent is delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Dosages

In general, an aminopyrimidine compound is administered in an amount of from about 10 mg to about 1000 mg per dose, e.g., from about 10 mg to about 20 mg, from about 20 mg to about 25 mg, from about 25 mg to about 50 mg, from about 50 mg to about 75 mg, from about 75 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 750 mg, or from about 750 mg to about 1000 mg per dose.

In some embodiments, the amount of an aminopyrimidine compound per dose is determined on a per body weight basis. For example, in some embodiments, an aminopyrimidine compound is administered in an amount of from about 0.5 mg/kg to about 100 mg/kg, e.g., from about 0.5 mg/kg to about 1 mg/kg, from about 1 mg/kg to about 2 mg/kg, from about 2 mg/kg to about 3 mg/kg, from about 3 mg/kg to about 5 mg/kg, from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, from about 40 mg/kg to about 50 mg/kg per dose, from about 50 mg/kg to about 60 mg/kg, from about 60 mg/kg to about 70 mg/kg, from about 70 mg/kg to about 80 mg/kg, from about 80 mg/kg to about 90 mg/kg, or from about 90 mg/kg to about 100 mg/kg, or more than about 100 mg/kg.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, multiple doses of an aminopyrimidine compound are administered. The frequency of administration of an aminopyrimidine compound can vary depending on any of a variety of factors, e.g., severity of the symptoms, etc. For example, in some embodiments, an aminopyrimidine compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in some embodiments, an aminopyrimidine compound is administered continuously.

The duration of administration of an aminopyrimidine compound, e.g., the period of time over which an aminopyrimidine compound is administer, can vary, depending on any of a variety of factors, e.g., patient response, etc. For example, an aminopyrimidine compound can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Routes of Administration

An aminopyrimidine compound is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. The compound can be administered in a single dose or in multiple doses.

An active agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

### Combination Therapies

In some embodiments, a subject treatment method will involve administering an effective amount of two or more different aminopyrimidine compounds. In some embodiments, a subject treatment method will involve administering a first aminopyrimidine compound for a first period of time; and administering a second aminopyrimidine compound for a second period of time, where the first and the second aminopyrimidine compounds are different from one another. In other embodiments, a subject treatment method will involve administering two different aminopyrimidine compounds substantially simultaneously.

In some embodiments, a subject treatment method will involve administering to an individual in need thereof an effective amount of an aminopyrimidine compound; and at least one additional agent that is effective for the treatment of an autoimmune disorder, or for reducing the risk of transplant rejection. In some embodiments, the at least one additional agent is other than an aminopyrimidine compound.

Those skilled in the art are aware of agents (other than aminopyrimidine compounds) that are suitable for treating autoimmune disorders. For example, agents that are suitable for treating Type 1 diabetes include insulin, including naturally occurring insulin, insulin analogs, and the like.

Insulin that is suitable for use herein includes, but is not limited to, regular insulin, semilente, NPH, lente, protamine zinc insulin (PZI), ultralente, insuline glargine, insulin aspart, acylated insulin, monomeric insulin, superactive insulin, hepatoselective insulin, and any other insulin analog or derivative, and mixtures of any of the foregoing. Insulin that is suitable for use herein includes, but is not limited to, the insulin forms disclosed in U.S. Pat. Nos. 4,992,417; 4,992,418; 5,474,978; 5,514,646; 5,504,188; 5,547,929; 5,650,486; 5,693,609; 5,700,662; 5,747,642; 5,922,675; 5,952,297; and 6,034,054; and published PCT applications WO 00/121197; WO 09/010645; and WO 90/12814. Insulin analogs include, but are not limited to, superactive insulin analogs, monomeric insulins, and hepatospecific insulin analogs.

Agents (other than aminopyrimidine compounds) that are suitable for reducing the risk of transplant rejection include, but are not limited to, cyclosporine (e.g., Sandimmune®, Neoral®); tracrolimus; corticosteroids (e.g., dexamethasone, methylprednisolone, methotrexate, prednisone, prednisolone, triamcinolone, etc.); sirolimus; mycophenolate mofetil; azathioprine; Daclizumab; Basiliximab; OKT3; rapamycin and derivatives thereof; leflunomide (or its main active metabolite A771726, or analogs thereof referred to as malononitrilamides); substituted xanthines (e.g. methylxanthines such as pentoxyfylline); brequinar; gusperimus; 6-mercaptopurine; mizoribine; chloroquine; hydroxychloroquine; and the like. Rapamycin derivatives include O-alkylated derivatives, particularly 9-deoxorapamycins, 26-dihydrorapamycins, 40-O-substituted rapamycins and 28,40-O,O-disubstituted rapamycins (as disclosed in U.S. Pat. No. 5,665,772) such as 40-O-(2-hydroxy)ethyl rapamycin (also known as SDZ-RAD), pegylated rapamycin (as disclosed in U.S. Pat. No. 5,780,462), ethers of 7-desmethylrapamycin (as disclosed in U.S. Pat. No. 6,440,991) and polyethylene glycol esters of SDZ-RAD (as disclosed in U.S. Pat. No. 6,331,547). In some embodiments, an aminopyrimidine compound, and one or more of the above-listed agents will be administered to a prospective transplant recipient, or to a transplant recipient.

### Subjects Suitable for Treatment

Subjects suitable for treatment using a subject method for treating an autoimmune disorder include individuals who have been diagnosed as having an autoimmune disorder; and individuals who are predisposed to developing an autoimmune disorder. Subjects suitable for treatment using a subject method for treating an autoimmune disorder also include treatment failure patients, e.g., individuals who have been diagnosed as having an autoimmune disorder, who have been treated with an agent other than an aminopyrimidine compound, and who have failed treatment with the agent other than an aminopyrimidine compound. Treatment failure patients include individuals who failed to respond to treatment; and individuals who initially responded to treatment, but subsequently relapsed.

Subjects suitable for treatment with a subject method for treating an autoimmune disorder include individuals who have been diagnosed with Type 1 diabetes mellitus. Such individuals include those having a fasting blood glucose level greater than about 126 mg/dL. Such individuals include those having blood glucose levels of greater than about 200 mg/dL following a two-hour glucose tolerance test (75 g anhydrous glucose orally). Subjects suitable for treatment with a subject method for treating an autoimmune disorder include individuals who have been diagnosed with Type 1 diabetes mellitus, and who retain at least some insulin-producing tissue function.

Subjects suitable for treatment with a subject method for treating an autoimmune disorder include pre-diabetic individuals who are at risk of developing Type 1 diabetes. Parameters associated with increased risk of developing Type 1 diabetes are known in the art; see, e.g., Diabetes Prevention Trial—Type 1 diabetes study group (2002) N. Engl. J. Med. 346:1685-1691 for examples of such parameters. Examples of individuals at risk of developing Type I diabetes include individuals having one or more of the following: circulating antibodies specific for islet cells (e.g., individuals with titers of 10 JDF units or higher); an HLA haplotype associated with increased risk of developing Type 1 diabetes; and a family history of Type 1 diabetes.

Subjects suitable for treatment with a subject method for treating an autoimmune disorder include individuals who have Type 1 diabetes, and who are recipients of a pancreatic islet cell transplant.

Subjects suitable for treatment with a subject method for increasing survival of a transplanted organ, tissue, or cells, methods of reducing the risk of transplant rejection, include prospective transplant recipients (e.g., individuals who are about to receive a transplant); and transplant recipients (e.g., individuals who have received a transplant. Subjects suitable for treatment with a subject method for treating GVHD include individuals who are transplant (graft) recipients.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Treatment of NOD Mice with Imatinib

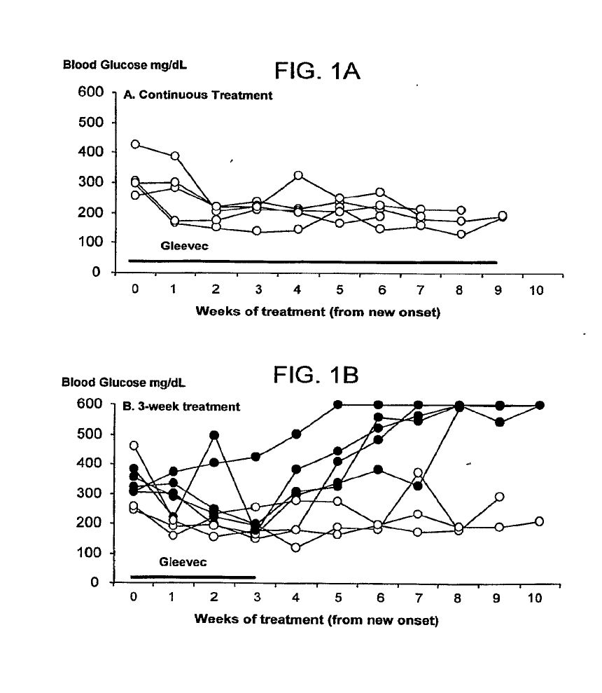

NOD mice were treated by gavage (oral) daily (M-F) with 50 mg/kg of commercially-available Gleevec suspended in peanut oil. Treatment was initiated at the time of disease onset (blood sugars above 250 mg/dl) and continued for the duration of the experiment. All of the treated mice went into remission and none relapsed during the course of the study (FIG. 1A). This contrasts with mice treated with peanut oil alone which does not induce remission.

Short-Term Treatment with Gleevec

Further studies were carried out to determine if Gleevec could be discontinued and maintain its efficacy. New onset mice were treated as above for only 3 weeks after onset and examined for disease recurrence (FIG. 1B). Greater than 90% of mice (7/8) went into remission during the 3 weeks of therapy. For the majority of mice, remission was short lived as 5/8 mice (FIG. 1B, solid circles) became hyperglycemic within 1-2 weeks after cessation of drug therapy. However, 3/8 mice (FIG. 1B, open circles) remained in remission more than 6 weeks after the last dose of Gleevec. Interestingly, mice treated with Gleevec maintained blood glucose levels of approximately 200 mg/dl consistent with blockade of continued aggressive autoimmunity but not regeneration of islets as has been postulated in anti-CD3 studies. These results show that protection from diabetes conferred by Gleevec is reliable and reproducible and suggest that the drug does not have to be given continuously to promote long-lasting remission. In addition, ongoing preliminary experiments suggest that Gleevec can effectively prevent disease in pre-diabetic animals. Ten week old NOD mice were treated daily with Gleevec (50 mg/ml) or peanut oil and followed for disease incidence. None of the Gleevec-treated and 50% of the peanut oil-treated mice were diabetic at 18 weeks of age. These results suggest Gleevec can function both in prophylaxis as well as therapeutically in T1D in NOD mice.

### Example 2

### Gleevec Prevention Studies

NOD Model

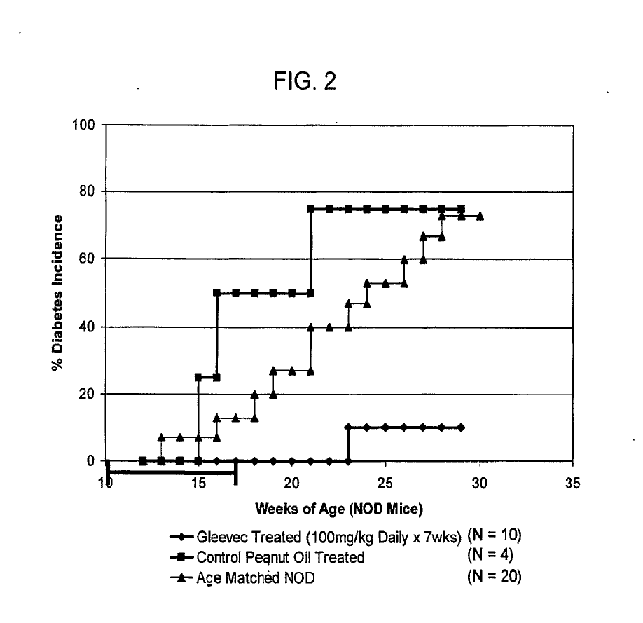

Pre-diabetic NOD mice were treated with Gleevec for 7 weeks starting at 12 weeks of age. The Gleevec was emulsified in peanut oil and given by gavage (100 mg/kg) daily. Control animals were treated with peanut oil alone. Mice were tested for diabetes based on blood glucose. Mice with two consecutive glucose readings of greater than 250 mg/dl were considered diabetic. As shown in FIG. 2, diabetes was prevented in 90% of mice, compared to peanut oil-treated control and untreated NOD mice.

Cyclophosphamide Model

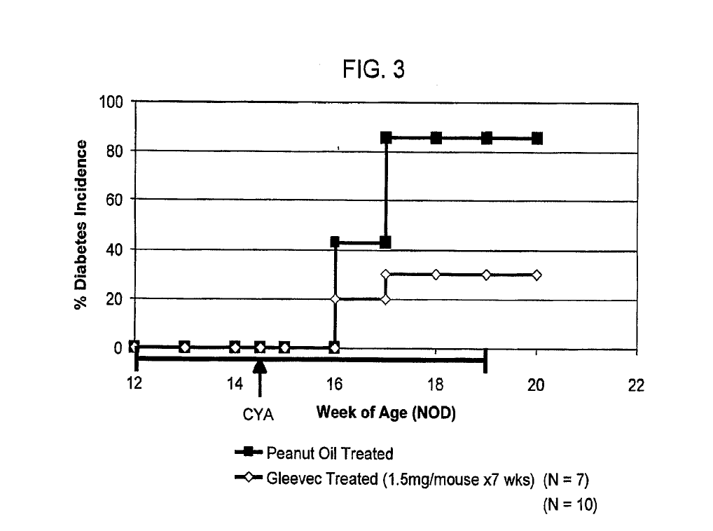

14.5 week-old NOD mice were injected intraperitoneally (i.p.) with cyclophosphamide (CY) (300 mg/kg) in conjunction with daily oral Gleevec therapy or control peanut oil. Gleevec treatment (1.5 mg/mouse/day) lasted 7 weeks. Mice were tested for diabetes based on blood glucose. Mice with two consecutive glucose readings of greater than 250 mg/dl were considered diabetic. As shown in FIG. 3, the incidence of diabetes was significantly reduced in Gleevec-treated mice as compared to peanut oil-treated control CY-treated NOD mice.

### Example 3

### Treatment of New Onset Diabetic NOD Mice

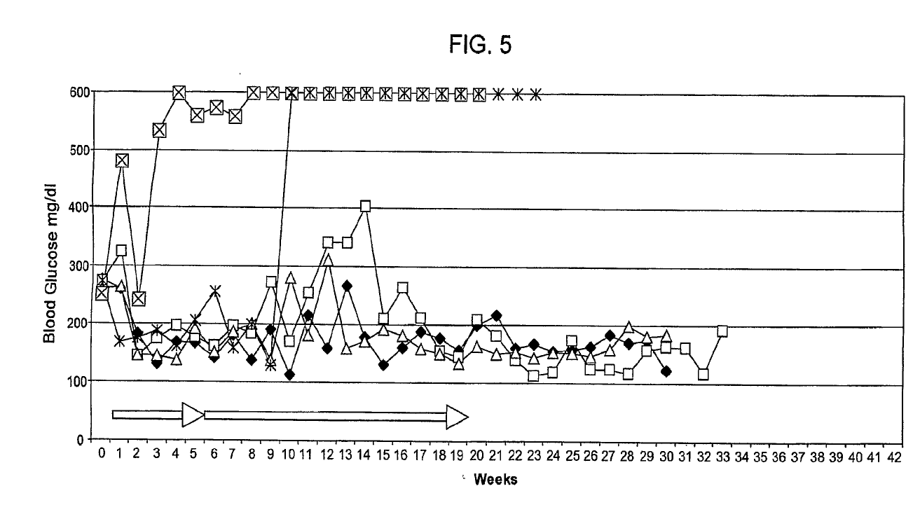

Diabetic NOD mice (250-350 mg/dL) were treated with Gleevec for a short course (3 weeks) or longer courses (8-10 weeks) daily 3 times per week (3×/wk); and blood glucose assessed as described in Example 2. The results are shown in FIG. 5. All treatments were effective in reversing diabetes in the majority of mice. However, long term maintenance (>20 week) of normoglycemia was less evident in short term 3 week treatment) than long term 8-10 week treated mice. The majority of the daily, 8-10 week-treated group remained normal glycemic for >2 months after ending therapy. Peanut oil treatment had no effect in reversing diabetes.

In another dosing regimen, NOD mice were treated with Gleevec (100 mg/kg) by daily oral gavage Monday through Friday for 2 weeks, followed by daily Gleevec (100 mg/kg) via oral gavage three times per week for 6 weeks. The data are presented in FIG. 6. Four of 5 mice went into disease remission. Three of the 5 mice maintained normoglycemia for the duration of the experiment (>30 weeks after onset).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating an autoimmune disease in an individual, the method comprising administering to the individual an amount of an aminopyrimidine compound.

2. A method of reducing the risk of rejection of a transplanted organ, tissue, or cells in an individual, the method comprising administering to the individual an amount of an aminopyrimidine compound that is effective to reduce the level of alloreactive T lymphocyte activity in the individual and reduce the risk of rejection of the transplanted organ, tissue, or cells.

3. The method of claim 1, wherein the aminopyrimidine compound is administered in an amount that is effective to reduce the severity of at least one symptom of the autoimmune disease.

4. The method of claim 1, wherein the autoimmune disease is selected from autoimmune hemolytic anemia, antiphospholipid syndrome, dermatitis, allergic encephalomyelitis, glomerulonephritis, Goodpasture's Syndrome, Graves' Disease, multiple sclerosis, myasthenia gravis, neuritis, ophthalmia, bullous pemphigoid, pemphigus, acute disseminated encephalomyelitis, polyendocrinopathies, purpura, Reiter's Disease, stiff-Man syndrome, inflammation, Guillain-Barre Syndrome, Type 1 diabetes mellitus, rheumatoid arthritis, autoimmune inflammatory eye disease, adult respiratory distress syndrome, inflammatory bowel disease, dermatitis, thrombotic thrombocytopenic purpura, Sjögren's syndrome, encephalitis, uveitis, leukocyte adhesion deficiency, psoriatic arthritis, progressive systemic sclerosis, primary biliary cirrhosis, pemphigus, pemphigoid, necrotizing vasculitis, systemic lupus erythematosus, polymyositis, sarcoidosis, granulomatosis, vasculitis, pernicious anemia, central nervous system inflammatory disorder, antigen-antibody complex mediated diseases, Hashimoto's thyroiditis, habitual spontaneous abortions, Reynard's syndrome, glomerulonephritis, dermatomyositis, chronic active hepatitis, celiac disease, tissue specific autoimmunity, degenerative autoimmunity delayed hypersensitivities, autoimmune complications of acquired immunodeficiency syndrome (AIDS), atrophic gastritis, ankylosing spondylitis and Addison's disease.

5. The method of claim 2, wherein the transplanted organ or tissue is selected from liver, heart, islet, pancreas, kidney, skin, and lung.

6. The method of claim 2, wherein the transplanted cells are selected from lymphocytes, dopamine-producing cells, bone marrow cells, stem cells, and blood cells.

7. The method of claim 2, wherein said administration is initiated before transplantation of the organ, tissue, bone marrow or cells.

8. The method of claim 1, wherein the aminopyrimidine compound is administered orally.

9. The method of claim 1, wherein the aminopyrimidine compound is administered continuously, three times daily, twice daily, once daily, every other day, three times per week, twice weekly, once weekly, once every other week, or once a month.

10. The method of claim 1, wherein the individual is a human.

11. The method of claim 1, wherein the autoimmune disease is Type 1 diabetes.

12. The method of claim 11, wherein blood glucose is maintained at normal level.

13. The method of claim 11, wherein the aminopyrimidine compound is administered continuously, three times daily, twice daily, once daily, every other day, three times per week, twice weekly, once weekly, once every other week, or once a month.

14. The method of claim 13, wherein the aminopyrimidine compound is administered over a period of time between one month and more than one year.

15. The method of claim 11, wherein the aminopyrimidine compound is administered for a first period of time and for a second period of time, wherein the first and second period of time are discontinuous.

16. The method of claim 1, wherein the aminopyrimidine compound is of the formula: 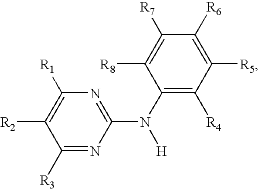
wherein
R₁ is 4-pyrazinyl, 1-methyl-1H-pyrrolyl, amino- or amino-lower alkyl-substituted phenyl wherein the amino group in each case is free, alkylated or acylated, 1H-indolyl or 1H-imidazolyl bonded at a five-membered ring carbon atom, or unsubstituted or lower alkyl-substituted pyridyl bonded at a ring carbon atom and unsubstituted or substituted at the nitrogen atom by oxygen,
R₂ and R₃ are each independently of the other hydrogen, lower alkyl, or pyrazinyl,
one or two of R₄, R₅, R₆, R₇, and R₈ are each independently nitro, fluoro-substituted lower alkoxy or a radical of the formula:
—N(R₉)—C(═X)—(Y)ₖ—R₁₀
wherein
R₉ is hydrogen or lower alkyl,
X is oxo, thio, imino, N-lower alkyl-imino, hydroximino or O-lower alkyl-hydroximino,
Y is oxygen or the group NH,
k is 0 or 1 and
R₁₀ is an aliphatic radical having at least 5 carbon atoms, or an aromatic, aromatic-aliphatic, cycloaliphatic, cycloaliphatic-aliphatic, heterocyclic or heterocyclic-aliphatic radical, or
one or two of R₄, R₅, R₆, R₇, and R₈ are each independently one of R₄, R₅, R₆, R₇, and R₈ is of the formula: 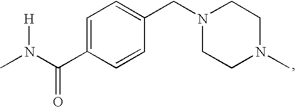
and the remaining groups R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterified hydroxy, free, alkylated or acylated amino or free or esterified carboxy,
or a salt of such a compound having at least one salt-forming group.

17. The method of claim 16, wherein the aminopyrimidine compound is of the formula:

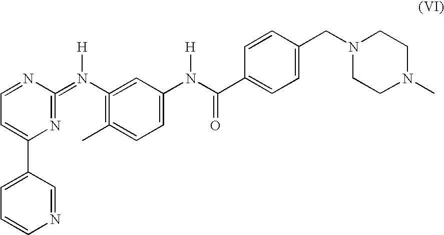

18. The method of claim 17, wherein the aminopyrimidine compound is of the formula:

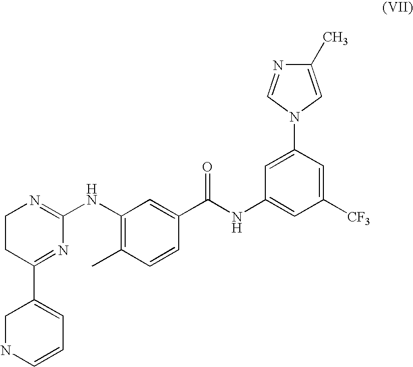

19. The method of claim 1, wherein the aminopyrimidine compound is of the formula: 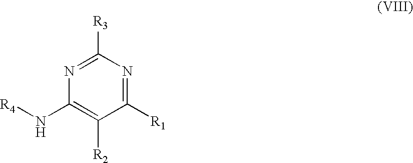
wherein R₁, R₂, and R₃ are each independently H, N, NH, hydroxyl, lower alkyl, or an aliphatic group having at least 5 carbon atoms, or an aromatic, aromatic-aliphatic, cycloaliphatic, cycloaliphatic-aliphatic, heterocyclic or heterocyclic-aliphatic group; and
R₄ is an aliphatic group having at least 5 carbon atoms, or an aromatic, aromatic-aliphatic, cycloaliphatic, cycloaliphatic-aliphatic, heterocyclic or heterocyclic-aliphatic group.

20. The method of claim 19, wherein the aminopyrimidine compound is of the formula:

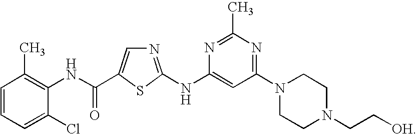

21. The method of claim 19, wherein the aminopyrimidine compound is of the formula: [chemical structure]
